(19) United States
(12) Patent Application Publication
(10) Pub. No.: US 2004/0143213 A1
(43) Pub. Date: Jul. 22, 2004

(54) Inertial drug delivery system

(75) Inventors: Ian Warwick Hunter, Lincoln, MA (US); Jane Hirsh, Wellesley, MA (US); Alison Fleming, North Attleboro, MA (US)

Correspondence Address: ROPES & GRAY LLP, ONE INTERNATIONAL PLACE, BOSTON, MA, 02110-2624, US

(73) Assignee: Collegium Pharmaceutical, Inc., Cumberland, RI

(21) Appl. No.: 10/712,734

(22) Filed: Nov. 12, 2003

Related U.S. Application Data
(60) Provisional application No. 60/425,549, filed on Nov. 12, 2002.

(51) Int. Cl. A61M 31/00

(52) U.S. Cl. 604/93.01; 604/173

(57) ABSTRACT

The systems and methods described herein include, inter alia, drug delivery devices that can provide doses of medicaments over a defined region of skin. Such systems and methods described herein may employ a hollow perforator that can receive a volume of drug and an actuator that can drive the perforator through a stroke cycle that accelerates the perforator and the volume of drug distally and then decelerates the perforator so that the volume of fluid is ejected out of the perforator.

### RELATED APPLICATIONS

This application claims the benefit of U.S. application Ser. No. 60/425,549, filed Nov. 12, 2002, the contents of which are incorporated by reference herein.

### BACKGROUND OF THE INVENTION

The systems and methods described herein relate, inter alia, to drug delivery systems, and to drug delivery systems that can precisely control the depth of the needle puncture as well as the volume of the percutaneously delivered drug.

Apparatus and systems for injecting drugs and other medicaments through or into the dermal regions of patients, both human and animal, are generally well-known in the art. Such systems typically employ needles having a wide range of lengths and diameters that pierce the skin and the subcutaneous tissue and enter underlying tissue and/or organs. A plunger or some analogous physical device within the system is operated to force a dose of medicine or other therapeutic agent in liquid form through the needle and into the patient. Such systems also generally include needles of very short lengths and sized to inject medicaments into the skin or into subcutaneous layers without necessarily reaching muscles or internal organs.

Other injection systems include jet injectors that spray a volume of medicine or other therapeutic agent at high pressure through a narrow aperture, without necessarily piercing the skin. This high pressure jet has a very narrow cross sectional area and, because of the high pressure, results in the injection of the medicament into or through the skin and/or subcutaneous and/or underlying tissue layers. Depth of injection depends on a number of factors including the viscosity of the medicament, the density of the tissues, and jet pressure.

The above-noted prior art systems suffer from a number of common disadvantages. The use of systems employing an exposed needle (“sharps”) presents a well-known safety hazard to medical personnel and others administering injections. In fact, the “Needlestick Safety and Prevention Act,” signed into law on Nov. 6, 2000, requires hospitals and health care facilities to use newer safety devices to reduce the number of needlestick injuries suffered by health care workers and patients. Pain associated with traditional injections via standard needles can cause patients to avoid visits with their health care providers. More importantly, successive stressful needle exposures can result in “needle phobia,” a medical condition that affects at least 10% of the population (Hamilton, J. G., The Journal of Family Practice, Vol. 41, No. 2, pp. 169-175). Injection site preparation requires extensive cleaning and sterilization so as to not result in infections. Even jet delivery systems, while ameliorating some of the above shortfalls, require carefully designed and expensive apparatus to prevent cross-contamination between patients when using the apparatus for multiple injections. Studies with one jet injection device showed poor patient compliance due to a complicated operating procedure and bruising and bleeding on injection (MacSwiney, B. P., et al, Archives of Disease in Childhood, Vol. 76, No. 1, pp. 65-67). Furthermore, the high pressure produced by jet injection may potentially cause damage to therapeutics with sensitive molecular structures such as proteins or other macromolecules.

The above-noted systems also fail to provide a convenient method of injecting drug over a defined area of skin. Single bolus injections rely on diffusion of drug from the injection site into the adjacent dermal regions, a process which is inefficient and time consuming. Creams and ointments, when applied topically, allow drug to be administered over a selected area but penetrate skin slowly or often fail to penetrate the outermost layer.

What is needed is a drug delivery system that can provide doses of medicaments over a defined region of skin using a microtube, or other small-scale needle-like device that provides only minimal puncture of the outermost dermal layer. Such a minute puncture avoids a number of issues related to injection site pain and potential for infection or other complications. Furthermore, such a drug delivery system needs to avoid sharps exposure when not operating to provide a safe operating environment for healthcare personnel.

### SUMMARY

The systems and methods described herein include, inter alia, drug delivery devices that employ a hollow perforator that can receive a volume of drug and an actuator that can drive the perforator through a stroke cycle that accelerates the perforator and the volume of drug distally and then decelerates the perforator so that the volume of fluid is ejected out of the perforator.

In one embodiment, the drug delivery systems include a permanent magnet producing a magnetic field. Within the magnetic field is a coil mounted on a flexible membrane or other flexure acting as a linear elastic element. Current flowing in the coil produces a proportional magnetic force (e.g., Lorentz force) that displaces the coil. The position of the coil and the flexure assembly upon which it is mounted varies with respect to the fixed magnet by magnitude and direction of current flow in the coil.

The actual position of the coil flexure assembly is transduced by a high resolution, high bandwidth linear displacement sensor (e.g., Hall effect sensor). The flexure position is determined and maintained under servo control by the control electronics which include, in some embodiments, a computer or microcomputer and associated analog and power control electronics well known in the art.

Mounted upon the flexure assembly is a drug reservoir containing at its distal end (i.e., the end away from the flexure) a microtube. Movement of the flexure by energizing and de-energizing the coil causes the drug reservoir and attached microtube to move rapidly in and out relative to the permanent magnet. When the permanent magnet and coil flexure assembly is mounted within a housing such that the microtube does not protrude from the casing until the coil is energized to displace the flexure outward, a completed drug delivery system is obtained.

In operation, the microtube is rapidly inserted into the patient's skin to a precise and adjustable depth by providing a current flow in the coil under control of the control electronics. The microtube is then withdrawn reversing the current flow in the coil and (optionally) increasing the magnitude of reverse current flow. Current flow reversal, especially at higher currents, can provide a very high acceleration of the flexure in an inward direction, e.g., opposite to the initial insertion of the needle. This opposing inward motion forces a small and controllable volume of the medicament mass contained in the interior volume of the microtube to pass through and be ejected from the tip of the microtube into the skin, where it remains. The ejection of the volume of the medicament mass results from the rapid movement of the microtube away from the skin while the medicament mass is still moving towards the skin. This “salt shaker” effect, whereby the drug in the microtube is left behind in the skin when the microtube/flexure assembly is rapidly withdrawn, will be referred to herein as “inertial delivery” or “inertial ejection.”

After inertial ejection of the medicament, the resulting negative pressure in the microtube caused by the inertial ejection, coupled with capillary forces, causes more medicament to be drawn into the microtube from the drug reservoir. This readies the microtube for the next insertion and subsequent inertial ejection. To equalize the pressure in the reservoir after ejection of the medicament, a relief valve or a gas source can be provided in connection with the reservoir.

The above described inertial drug delivery system finds application in situations where it is preferred that a drug dosage be delivered over a relatively large area of skin, such as to avoid the pain and discomfort associated with a single large injection or to provide a relatively uniform concentration of drug within a given area of skin.

In further embodiments of the present invention, the housing containing the above-described permanent magnet, control electronics, and flexure/drug reservoir/microtube assembly may be equipped to provide position feedback of the microtube, drug reservoir, and/or flexure. For example, the positional sensor, e.g., a piezoelectric motion sensor, may determine or monitor the position or movement of the microtube in its motion toward or away from the skin.

Another type of positional sensor may determine or monitor the position or movement of the housing on the surface of the skin. Such position feedback may be supplied by, for example, an optical sensor, or rotational sensors that are connected to wheels that roll on the surface of the skin. The sensors may be configured to provide either one-dimensional or two-dimensional position feedback. The actual motion of the housing relative to the skin surface and/or external location reference points (e.g., skin markers delimiting the treatment area) is thus sensed by conventional positioning means, for example, those used in optical computer “mouse” pointing devices. With knowledge of the two dimensional position on the skin surface, the device may be triggered to inject every millimeter or every few millimeters (i.e., in a grid pattern). Alternatively, the device may be configured to inject at regular time intervals such that the distance between individual insertions of the microtube is controlled manually by moving the housing relative to the skin surface.

In some embodiments, the system comprises a positioning means for moving the actuator relative to the skin of the patient, wherein the positioning means further comprise a sensing means. One such embodiment of a sensing means may be an optical detection system configured to detect a mark. The mark may be made by the care giver to outline or indicate an area for treatment. Alternatively, the mark may be caused by the condition being treated. Other such embodiments may use sensing means comprising impedance, temperature, pH, or chemical sensors, or any other such sensor known to those in the art.

### BRIEF DESCRIPTION OF THE DRAWINGS

The present disclosure may be better understood and its numerous features and advantages made apparent to those skilled in the art by referencing the accompanying drawings.

FIG. 1 is a cross-sectional view of a drug delivery system according to one embodiment of the present invention.

FIG. 2 is a schematic diagram of a scan across patient's skin with the embodiment of FIG. 1.

FIG. 3 is a graph depicting the vertical displacement of a perforator with respect to time of an inertial drug delivery system of the present invention.

FIG. 4 is a graph depicting the vertical displacement of a perforator of an inertial drug delivery system with respect to the system's position on the surface of the skin.

FIG. 5 illustrates the various positions of a reservoir and perforator at different points during the delivery of the medicament.

FIG. 6 shows four examples of a perforator.

FIG. 7 depicts a perforator and reservoir configuration of an embodiment of the invention in which the microtube and reservoir are in fluid connection by means of a flexible tube.

FIG. 8 is a cross-sectional view of an inertial drug delivery system according to an embodiment of the invention.

FIG. 9 is a cross-sectional view of an inertial drug delivery system according to an embodiment of the invention.

The use of the same reference symbols in different drawings indicates similar or identical items.

### DETAILED DESCRIPTION

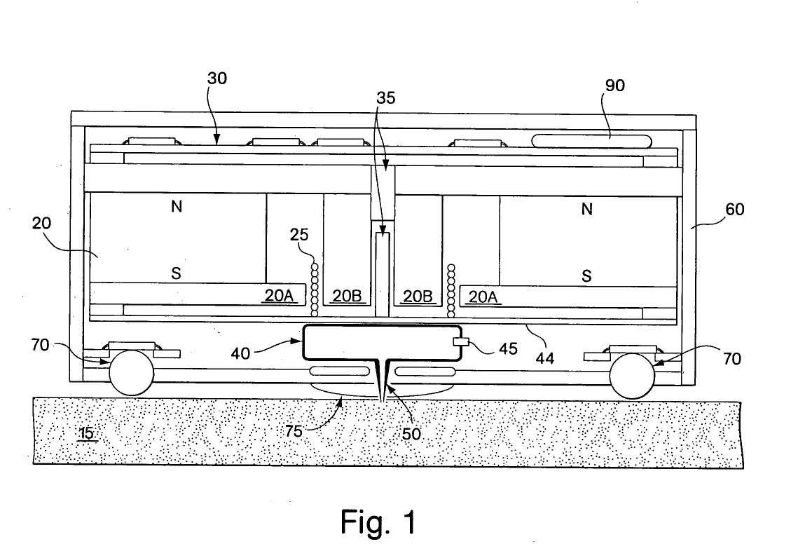

As shown in cross-section in FIG. 1, one exemplary embodiment of an inertial drug delivery system includes a housing 60 placed in contact with the skin 15 of a patient. A moveable support, such as ball bearings and two-axis position sensor assemblies 70, hold the housing 60 at a slight elevation above skin 15. The exemplary housing 60 is shown in the cross-section of FIG. 1 as generally rectangular, although one of ordinary skill in the art will recognize that a housing can be cylindrical, hemispherical, or of any shape sufficient to hold and protect its contents.

In some embodiments, a bulge area 75 may protect microtube 50 by ensuring that the sharp point of the microtube does not project outside the housing until the device is activated. The bulge area 75 may be made of any biocompatible material, such as metal, ceramic, or polymer. Examples of useful metals include titanium and stainless steel. A further safety interlock system maybe incorporated, using conventional sensing and electronics, to ensure that microtube 50 cannot be extended outside of the housing, or at least not outside the bulge area 75, unless the device is in controlled positive contact with the patient and/or under positive control by appropriately trained personnel.

In a further alternate embodiment, the bulge area 75 may be configured to stretch the skin 15 at the area of contact in order to facilitate rapid penetration by the microtube 50, discussed further below.

Although a perforator such as a microtube having a small length (on the order of 5 millimeter or less with a cross-section of the orifice on the order of 20 to 800 micrometers in diameter) and being surgically sharp at its penetrating or piercing end is described, those skilled in the art will realize that conventional small needles and other small perforators other than a microtube can be used. Such small needles may be fabricated by any number of conventional methods well-known in the art, including micromachining, chemical processes, microinjection molding, 3-dimensional stereolithography, electrochemical etching, direct metal forming, or glass processing as known in the art. Accordingly, the invention is not limited to any particular type of perforator. Perforators such as microtubes or conventional small needles utilized in the present invention will generally be in the range of about 200 micrometers to about 5 millimeters in length, preferably between about 200 micrometers to about 1 millimeters, about 250 micrometers to about 2 millimeters, or more preferably between about 300 micrometers to about 0.8 millimeters. Perforators utilized in the present invention will generally have an orifice in the range of about 20 micrometers to about 800 micrometers in diameter, preferably about 50 micrometers to about 500 micrometers, and more preferably about 100 micrometers to about 300 micrometers. One of skill in the art would understand that the length and the orifice size of the microtube may be varied and can be selected as appropriate for the application. The length and the orifice size of the perforator may also depend on whether intramuscular, intradermal, or subcutaneous injection is desired.

Located within the housing 60 is a magnet 20, in one embodiment having a ring or toroidal shape. Magnet 20 includes magnet poles 20A and 20B which may be arranged in any of several orientations known in the art. The magnet 20 is preferably a permanent magnet, for example, a rare-earth based magnet, made of for example samarium-cobalt, neodymium iron boron, or similar high energy density magnets producing high magnetic fields in the order of about 0.1 Tesla to about 2.5 Tesla. Alternatively, the magnet can also be an electromagnet. A magnet coil 25 extends into the magnet gap between poles 20A and 20B. As shown in the cross-section view of FIG. 1, a suspension flexure 44 is mounted upon the permanent magnet 20 and operatively connected to the coil 25. A current is supplied to the coil 25 by the control electronics 30 through conventional electrical connections (not shown). The produced electromagnetic force (Lorentz force) causes the flexure 44 to move either towards or away from magnet 20, depending on the direction of the supplied current.

The position of the flexure 44 can be measured by a displacement sensor 35, such as a Hall Effect sensor. Other forms of displacement sensors and position transducers suitable for use in this application, such as capacitive and optical sensors, can also be employed. Other examples include an impedance sensor, a temperature sensor, and a pH sensor. Accordingly, the invention described is not limited to any one particular type displacement sensor.

A drug reservoir 40 is attached to the flexure 44 by any number of means known in the art and contains a quantity of medicine or other therapeutic agent, preferably (although not exclusively) in liquid form. Reservoir 40 also includes or is connected to a microtube 50, which in its extended (operating) position extends outward from the reservoir through an opening in the surface of housing 60 which contacts the skin of a patient. The connection between the microtube and the drug reservoir can be achieved by a variety of means. The microtube 50 and drug reservoir 40 can be welded or otherwise irreversibly connected or, alternatively, connected via a reversible mechanism such as a threaded screw or interlocking snap. The microtube and drug reservoir can also be connected via a flexible tube. When the bulge area 75 is present, microtube 50 likewise extends through bulge 75 when operating. As noted above, when in either its quiescent (un-energized) state or retracted state, the microtube 50 does not extend beyond the outside edge of housing 60 or (when present) bulge area 75.

When the control electronics 30 energizes the coil 25, with current flowing through the coil 25 in a first direction, the suspension flexure 44 is urged toward the skin, causing the microtube 50 to project outwardly from the bulge 75 and penetrate the skin 15. On reversal of the current flow in coil 25, by means of control electronics and current shaping circuits well known in the art, the suspension flexure 44 is urged away from the skin, potentially with a high acceleration as determined by the current flowing through the coil 25 in the reverse direction. The sudden stop coupled with the rapid acceleration of drug reservoir 40 and microtube 50 away from the skin will force a small amount of medicament to exit the tip of the microtube 50 and remain within the skin 15. This result is a direct effect of the inertia of the medicament developed during insertion and withdrawal of the microtube 50. The rapid reversal of microtube motion causes the medicament mass to overcome any frictional or capillary forces which may cause it to remain in microtube 50, and to be ejected from the microtube 50, remaining inside skin 15.

In some embodiments, computer or manual control may be used to move housing 60 relative to the surface of the skin. This motion should not occur until microtube 50 is retracted substantially completely from skin 15. Such control may be provided by control electronics 30 operating in conjunction with an optional on-board power source 90 (such as a battery). Alternatively, the control electronics 30 could be connected to a power mains and/or directed by an external controller (not shown) to move or operate (inject) in accordance with the directives of a person or a sequence of programmed actions.

In other embodiments, the system is designed to deliver multiple injections while the housing remains in one location. The perforator moves within the housing along the plane of the skin to enter the skin in different locations in a predetermined pattern, e.g., a grid or concentric rings. For example, a housing may be placed and held on one location of a patient's back for a period of time. During that period, the perforator is inserted into the skin multiple times in, for example, a grid formation. At the end of the period, which may be indicated by a sound or light, the housing may be moved to another location on the patient's back to deliver additional medicament.

The quantity of drugs delivered in each microtube insertion may be determined by the internal volume of the microtube 50, which may be in the range of 0.5 nanoliter to 10 microliters, or by the velocity and acceleration of the withdrawal, or a combination of various factors. A perforator or microtube may have an internal volume of about 0.5 nanoliter to about 10 microliters, preferably of about 1 nanoliters to about 5 microliters, and more preferably of about 2 nanoliters to about 2 microliters, however, other volumes are possible and can be selected as appropriate for the application. This volume may also depend on factors such as the dose of drug required for effect, the therapeutic index of the drug, and the concentration of drug in solution.

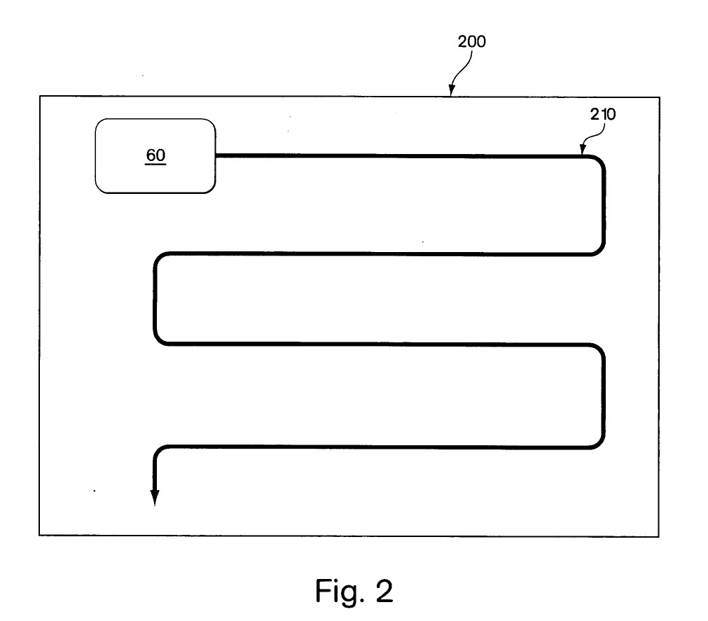

FIG. 2 shows a system whereby housing 60 is scanned across the skin surface 200 to produce a pattern of injections. Path 210 represents one of several potential paths of motion of housing 60. Paths can be chosen, in some embodiments, by operator or computer selection depending on the contemplated dosing and/or medical needs of the patient.

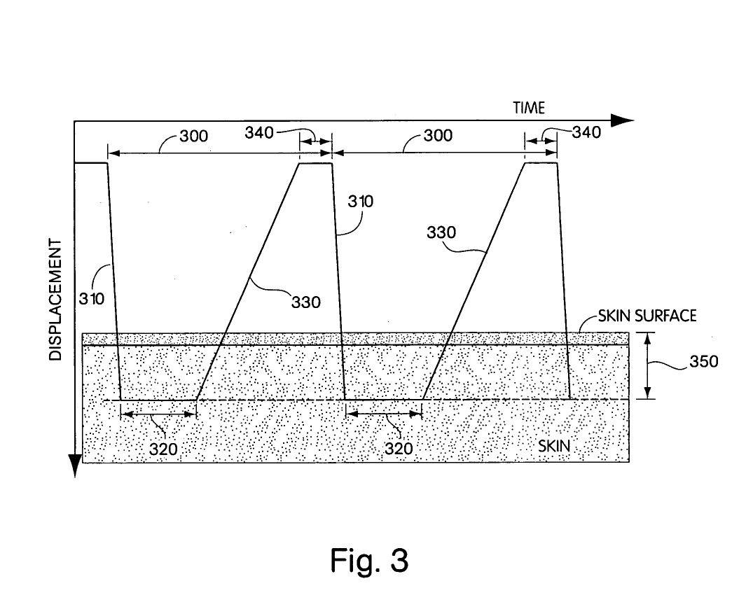

FIG. 3 depicts a graph showing on the y-axis displacement and on the x-axis time. The graph of FIG. 3 shows the vertical displacement of a perforator of a device with respect to time. A full cycle 300 includes insertion velocity 310, dwell time 320, withdrawal velocity 330, and rest time 340. Insertion velocity 310 represents the vertical position of a perforator over the period of insertion. Dwell time 320 represents vertical position of a perforator at a desired or predetermined insertion depth over a time period. Withdrawal velocity 330 represents the vertical position of a perforator over a time period of withdrawal. Rest time 340 represents vertical position of a perforator at an end position over a time period. In the depicted embodiment, insertion velocity 310 is over a shorter period of time than withdrawal velocity 330 and dwell time 320 is over a longer period of time than rest time 340. In other embodiments, insertion velocity 310, withdrawal velocity 330, dwell time 320, and rest time 340, along with the time period in which cycle 300 occurs and insertion depth 350, may be varied. For example, insertion velocity 310 may occur over a longer or same period of time than withdrawal velocity 330, and rest time 340 may occur over a longer or same period of time than dwell time 320. The duration of the cycle 300, insertion velocity 310, dwell time 320, withdrawal velocity 330, and rest time 340 may vary according to the application at hand and appropriate durations will be known to those of skill in the art. In certain practices, the durations are about 1.0 second, 0.1 seconds, 0.01 seconds, 0.001 seconds, but other durations are possible.

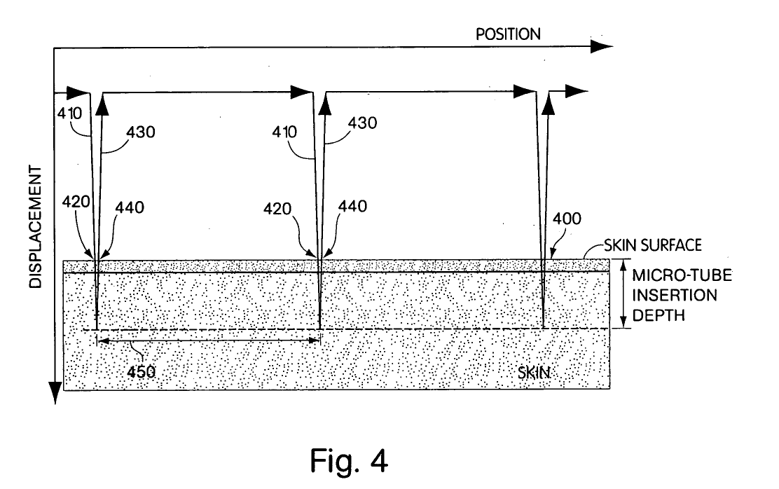

FIG. 4 depicts a graph showing on the y-axis vertical displacement and on the x-axis horizontal displacement. The graph of FIG. 4 shows the vertical displacement of a perforator of an embodiment according to the present invention with respect to different positions along the surface of the skin 400. The movement across the skin of the perforator between insertion point 420 and withdrawal point 440 is minimal, whereas the movement across the skin between the different insertion points 450 may be substantially longer. Insertion 410 and withdrawal 430 of the perforator may occur with almost no movement along the surface of the skin 400. The distance between insertion points 450 may be any distance required by a particular treatment situation. For example, insertion points 420 may be separated by a distance of about 100 cm, 50 cm, 10 cm, 1 cm, 1 mm, 0.1 mm, 0.01 mm, 0.001 mm, but other distances are possible and can be selected as appropriate for the application. This distance may also depend on whether intramuscular, intradermal, or subcutaneous injection is desired.

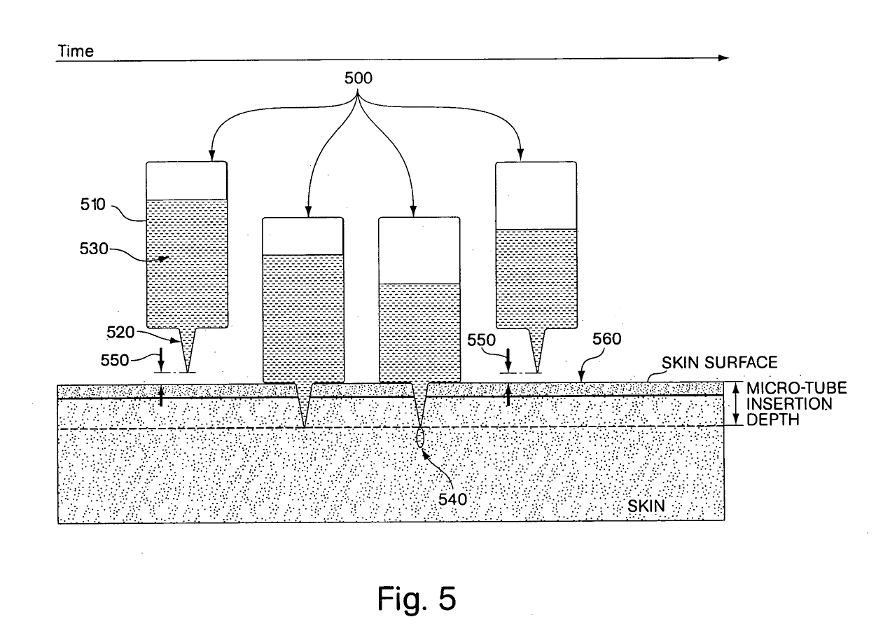

FIG. 5 shows the position of device 500, which includes a reservoir 510 and perforator 520, of an embodiment of the invention at different points of a delivery cycle. Reservoir 510 contains medicament 530 and is in fluid connection with perforator 520. Device 500 is located at the beginning at distance 550 from the skin surface 560. Device 500 is accelerated toward the skin surface 560 and perforator 520 is driven into the skin. The velocity of insertion in the skin will be high and occur over a short period of time. Device 500 is stopped abruptly by the skin. The momentum of medicament 530 causes it to continue to move in the same direction after device 500 is stopped and ejects a small volume of medicament 540 through perforator 520 into the skin. Device 500 is then withdrawn to distance 550 at which point the cycle can begin again.

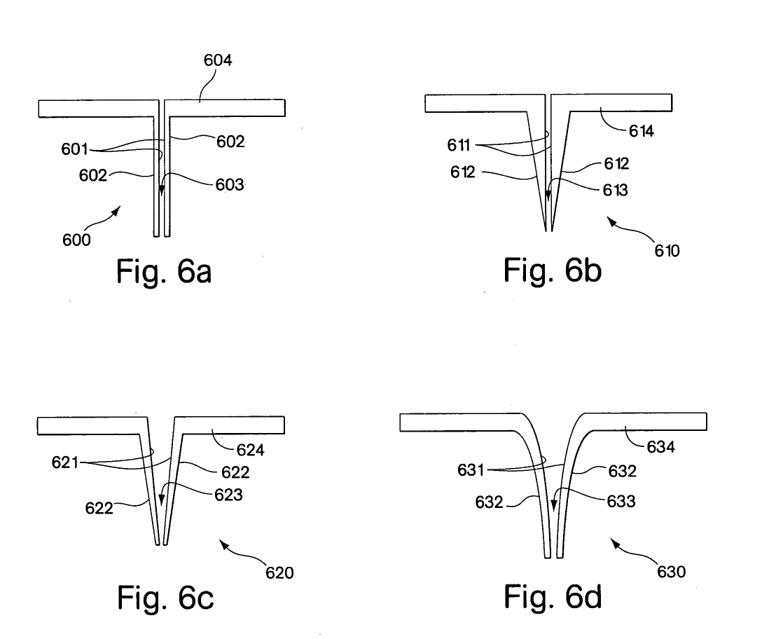

FIGS. 6A-6D show the cross sectional views of four different perforator designs, respectively, microtubes 600, 610, 620, and 630. FIG. 6A depicts microtube 600. Microtube 600 has inner wall 601 and outer wall 602. Inner wall 601 is substantially parallel to outer wall 602. Both inner wall 601 and outer wall 602 are substantially perpendicular to base 604. Channel 603 has a substantially uniform diameter.

FIG. 6B depicts microtube 610. Microtube 610 has inner wall 611 and outer wall 612. Inner wall 611 is substantially perpendicular to base 614. Outer wall 612 has an angle relative to base 614. Channel 613 has a substantially uniform diameter.

FIG. 6C depicts microtube 620. Microtube 620 has inner wall 621 and outer wall 622. Both inner wall 621 and outer wall 622 have an angle relative to base 624. Although inner wall 621 and outer wall 622 are shown as having two different angles, one of skill in the art would understand that the inner and outer walls of a perforator may have the same angle. Channel 623 has a diameter decreasing from the base 634 to tip of microtube 620.

FIG. 6D depicts microtube 630. Microtube 630 has inner wall 631 and outer wall 632. Both inner wall 631 and outer wall 632 curve along the length of microtube 630. Channel 633 has a diameter decreasing non-linearly from base to tip of microtube 630. The perforator used in the drug delivery system may be selected from one of these microtube designs or may be any other small, hollow perforator.

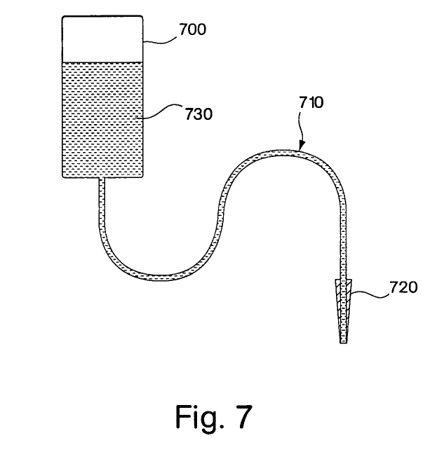

FIG. 7 shows an alternative configuration for a microtube 720 and reservoir 700 in which the reservoir 700 contains medicament 730 and is connected to the microtube 720 by a flexible tube 710. The flexible tube 710 may be of any length to permit the placement of the reservoir 700 at any desired location. Medicament 730 may be delivered by actuating reservoir 700 and/or microtube 720. In one embodiment, a reservoir 700 may be in close proximity to the microtube 720 connected by a short flexible tube. In another embodiment, a reservoir 700 may be suspended from a support or resting on a shelf connected to the microtube 720 by a long flexible tube.

Multiple injections to deliver a therapeutic dose of medicament are feasible and may be required in certain applications. The number and location of these injections can vary according to the type of medicament used and the desired effects. Likewise, the rate of injection can be varied to suit the type and quantity of medicament to be delivered. It is anticipated that typical rates will be 1 to 20 times per second, and for certain applications may be up to 500 times per second. The choice of number of injections and patterns of injections is readily determined by the total amount of medicament desired and the well-known drug absorption qualities of the area of skin so affected. Additionally, the depth of insertion of the microtube, and hence the drug deposition depth, will be selected based on factors such as the type of medicament and the desired effects. While it is recognized that achieving minimal puncture of the outermost layers of skin (approximately 150 micrometers or less) will avoid contact with the nerves and thus minimize discomfort to the patient, the depth of needle insertion may range from 20 micrometers to 4 millimeters. Variability of insertion depths may be achieved by providing for adjustment of the displacement of the flexure, or, alternatively, the insertion depth may be fixed for a given device provided that the device is used only in conjunction with appropriate medicaments. The device may incorporate features that distract patients from any discomfort from microtube insertion, including but not limited to local heating or cooling of the skin, vibrations, or other sensory input.

In general, the invention is directed to a medicament delivery system with an actuator, a flexure mounted on the actuator, and a fluid reservoir disposed on the flexure in fluid connection with at least one hollow perforator and including a medicament, wherein the perforator pierces the skin of a patient and a subsequent withdrawal of the perforator results in the delivery of the medicament.

The delivery of the medicament may be inertial delivery. The delivery of the medicament may also be due to a pressure inside the reservoir greater than the pressure outside of the reservoir, e.g., in the air. The delivery of the medicament may result from a combination of factors including the aforementioned factors.

The flexure is capable of movement in response to the actuator. The fluid reservoir has a proximate and a distal surface. The fluid reservoir may be fixedly attached to the flexure by, for example, the proximate surface. The fluid reservoir may be permanently or non-permanently attached to the flexure. The movement of the flexure in the distal direction may cause the reservoir and the perforator to pierce the skin.

In a further embodiment, the system comprises a plurality of perforators. In another embodiment, the system may comprise a plurality of reservoirs in fluid connection with at least one perforator. The plurality of reservoirs and perforators may enable multiple deliveries of one or more medicaments per injection.

In a still further embodiment, the delivery system comprises a housing having an aperture therein. The housing may enclose the actuator, the flexure, and the fluid reservoir and be configured to permit the perforator to extend distally from the housing in response to the actuator moving the flexure. Further the housing may conceal the perforator when the actuator is not moving the flexure or when the device is in its resting state. The housing may be made of any one of a number of materials such as, but not limited to: metals like stainless steel, aluminum, or titanium; ceramics; polymers; composites; glasses; or a combination of a number of different materials.

The housing may further comprise a bulge area surrounding the aperture. The bulge area may extend distally from the aperture to form a raised surface for contacting the skin. The bulge area can be made of a rigid or resilient material, preferably a material that is bio-compatible with the skin. The bulge area may be configured to stretch the skin at its point of contact. This may be accomplished by having the three-dimensional the shape of the bulge area such that it pushes or pulls the skin to provide a more rigid surface to facilitate piercing of the skin. Examples of structures to stretch skin include flanges sitting on the bulge area that move radially apart to stretch the skin the flanges contact or a circular ridge surrounding the aperture that holds the skin substantially motionless. The bulge area may be made of any one of a number of materials such as, but not limited to: metals like stainless steel, aluminum, or titanium; ceramics; polymers; composites; glasses; or a combination of a number of different materials.

It is anticipated that the energy required to drive the actuator and any electronics may come from batteries (e.g., alkaline or zinc air). However, in some embodiments it may be desirable to use rechargeable batteries and in other embodiments it may be beneficial to use rechargeable super capacitors as an energy source. In still other embodiments an external energy source may be appropriate.

All or some components of the present invention may be disposable, that is, they may be designed for a single application on a single patient. The microtube, drug reservoir, and any components that may come into contact with the patient will be disposable in most cases; this will eliminate the possibility of contamination between patients. In some embodiments the housing, actuator and electronics will be re-usable. In such embodiments the reservoir/microtube subassembly may be fixed within the housing via a reversible mechanism such as a threaded screw, an interlocking snap or a bayonet joint.

The order in which the steps of the present method are performed is purely illustrative in nature. In fact, the steps can be performed in any order or in parallel, unless otherwise indicated by the present disclosure.

While an actuator system consisting of, in one embodiment, a fixed magnet and coil in a solenoid-like configuration is shown, those of ordinary skill in the art will recognize that numerous other mechanical or electromechanical actuation systems can also be used to move a microtube with precision. Examples of such systems are found in conventional voice coils, speakers, transducers, solenoid systems, mechanically-linked rams, hydraulic or pneumatic systems, spring-loaded push buttons and the like now known or foreseeable. Accordingly, this invention is not limited to any particular form of actuator.

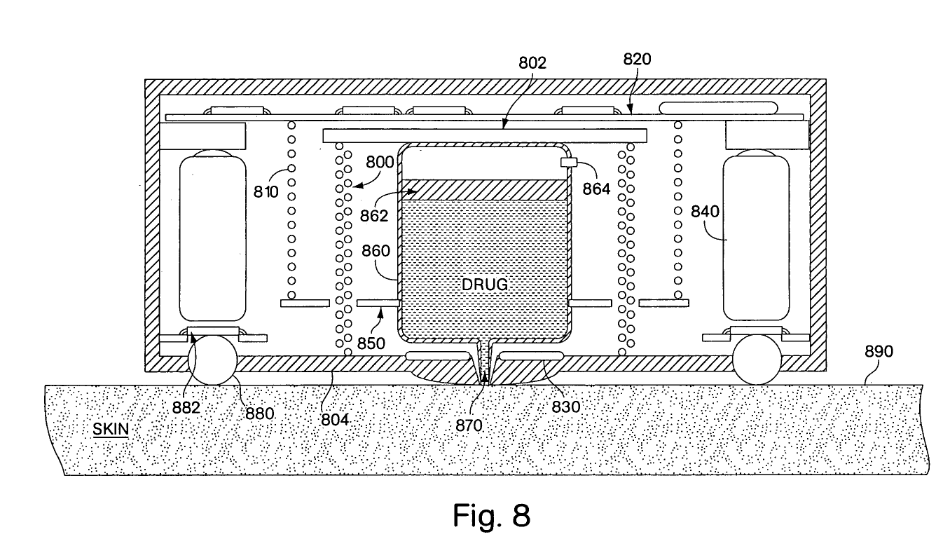

Shown in cross-section in FIG. 8 is an embodiment of the drug delivery system with an alternate means for driving the actuator. This embodiment uses the combination of shape memory alloy fibers 800, control electronics 820, and springs 810 to effect drug delivery. Reservoir 860 is mounted on flexure 850 and platform 802. Return extension springs 810 are attached at one end to control electronics 820 and at the other end to flexure 850. Shape memory alloy fibers 800 are attached at one end to platform 802 and at the other end to housing 804. Fibers 800 contract when an electrical pulse is delivered to them from the control electronics 820. The control electronics 820 may be powered by a battery 840. The contraction of fibers 800 drives the reservoir 860 and microtube 870 toward the skin 890 and microtube 870 pierces the skin 890. The contraction of fibers 800 also extends springs 810. The reservoir 860 and microtube 870 stop moving toward the skin when the reservoir 860 hits the end stop 830. Upon stopping of reservoir 860 and microtube 870, a small volume of medicament is delivered into the skin 890. The extended springs 810 generate an opposing force that returns the shape memory alloy fibers 800 to their original position and hence retracts the reservoir 860 and microtube 870 from the skin 890. The shape memory alloy 800 may be configured in a spiral to achieve the desired contractile displacement. Reservoir 860 further includes plunger float 862 and relief valve 864. A moveable support, such as ball bearings 880 and two-axis position sensor assemblies 882, hold the system at a slight elevation above skin 890.

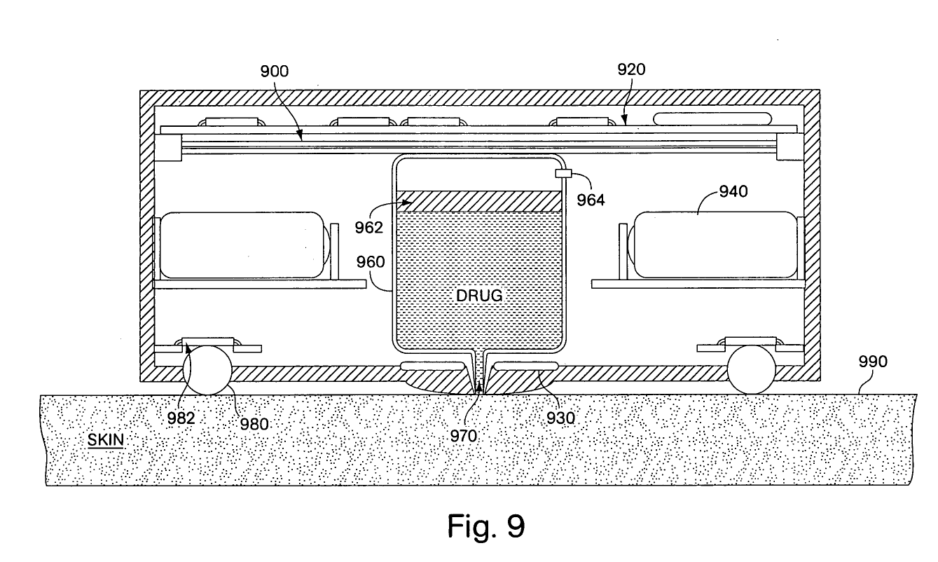

Shown in cross-section in FIG. 9 is another embodiment according the present invention with an alternate means for driving the actuator. Reservoir 960 is mounted on piezoelectric bimorph actuator 900. The piezoelectric bimorph actuator 900 is activated when a positive voltage pulse is delivered from the control electronics 920. The control electronics 920 may be powered by a battery 940. This actuation drives the reservoir 960 and microtube 970 toward the skin 990. The reservoir 960 and microtube 970 stop moving towards the skin when the reservoir 960 hits the end stop 930. Upon stopping, a small volume of medicament is delivered into the skin through microtube 970. The piezoelectric bimorph actuator 900 is then supplied with a negative voltage pulse, which cause the actuator 900, the reservoir 960 and microtube 970 to retract and return to their original position. Reservoir 960 further includes plunger float 962 and relief valve 964. A moveable support, such as ball bearings 980 and two-axis position sensor assemblies 982, hold the system at a slight elevation above skin 990.

Furthermore, while a flexure mechanism is generally described, such flexures are not limited to any particular form, and all flexures generally known in the art are suitable. In one embodiment, a diamond-coated titanium membrane attached as shown in FIG. 1, forms a preferred embodiment of flexure 44. However, other linear or non-linear elastic materials and combinations of such materials having sufficient strength to withstand repeated flexing by the actuator are equally suitable. The flexure may be a membrane made of materials such metals, polymers, ceramics, etc. Alternatively, the flexure may be substituted by a component for guiding the motion of the reservoir such as sleave bearings, roller or ball bearings, air bearings, magnetic bearings. Accordingly, the invention is not limited to a particular flexure type or configuration, or even to a flexure.

Drug reservoir 40 is shown in cross-section as the generally rectangular structure having a relief valve 45 and microtube 50. One of ordinary skill in the art will recognize, however, that drug reservoirs of any cross-section or overall volume, including tubular, cylindrical and conical forms to name just a few, may be used. The total reservoir volume will generally be in the range of about 10 microliters to about 50 milliliters, preferably between about 50 microliters to about 50 milliliters, 100 microliters to about 25 milliliters, and more preferable about 100 microliters to about 5 milliliters. This is the preferred by inventors. Materials for the fabrication of the drug reservoir will preferably be inert. Examples of such materials include, but are not limited to, metals, glass, glass coated polymer materials, polymer materials and ceramic materials. Such drug reservoirs generally are only constrained by the need to fit within housing 60 and to have mounted on them microtube 50. Microtube 50 should be mounted so as to prevent movement relative to flexure 44, so that the movement of flexure 44 is directly translated into the movement of microtube 50. While relief valve 45 is only shown in one location, several relief valves may be used in order to prevent “vapor lock” or other restriction in drug reservoir 40 that would prevent refilling of the medicament channel within microtube 50.

An alternative embodiment, the reservoir may include an ampoule within a reservoir housing such that the ampoule can be broken open at the time of use. In another embodiment, the reservoir may include two or more ampoules containing different drugs which mix upon the breaking of the ampoules and are delivered together. The ampoule may be made from inert materials such as glasses or polymers. This may be useful for storing medicaments that may readily decompose when exposed to air and for keeping the medicament sterile.

In some embodiments a plunger or float is present within the drug reservoir. The plunger or float serves to constrain the medicament solution as the volume of solution is incrementally reduced during regular use. The plunger or float will progressively move down the reservoir, toward the microtube, as the drug is discharged into the skin, thereby constraining the drug solution to the microtube end of the reservoir. The presence of such a plunger will allow the device to be used at arbitrary angles; without such a plunger, the location of the drug solution within the reservoir will be under the control of gravity and may not necessarily be localized at the opening to the microtube.

In an alternate embodiment the reservoir may be in fluid connection with a gas source to provide pressure to supplement the inertial force for delivering the medicament. In one such embodiment, an electrolysis system may be used to generate gas from water. The gas source may include a gauge or valve to measure and/or control the pressure provided to the reservoir. The gas pressure, or alternatively an aerosol, nitrogen flush, or some other material providing extra force, can be used to deliver the medicament, such as those of a specific gravity, density, or viscosity greater than water, for example, suspensions, emulsions, gels, and oils.

A relief valve may be unnecessary if the formulation of the drug and the balance of gas volume within drug reservoir 40 are such that capillary action can refill microtube 50 after each initial injection cycle. Accordingly, the invention is not limited by the presence or number of relief valves.

Control electronics 30, as contemplated herein, include generally hardware, software or any combinations thereof, as those terms are currently known in the art. In particular, the control system may be implemented in software, firmware, or microcode operating on a computer or computers or any type, either standing alone or connected together in a network of any size. Additionally, software embodying control algorithms for use in the present invention may comprise computer instructions in any form (e.g., source code, object code, interpreted code, etc.) stored in any computer readable medium (e.g., ROM, RAM, magnetic media, punched tape or card, compact disc in any form, DVD, etc.). Furthermore, such control software may also be supplied in the form of a computer data signal embodied in a carrier wave, such as that found within the well-known web pages transferred among devices connected to the Internet. Accordingly, the computer control aspects of the present invention are not limited to any particular platform.

### APPLICATIONS

The aim of drug therapy is to prevent, cure or control various disease states for a person in need. To achieve this goal, an adequate dose of a therapeutic agent must be delivered to the target tissues so that the desired therapeutic response is obtained. The route of administration is determined primarily by the therapeutic objective (e.g., ultimate site of activity, rate of therapeutic onset, duration of activity) and the properties of the agent (e.g., stability, solubility). In general, therapeutic agents can be administered at their site of action (“local delivery”) or into the blood where they circulate throughout the body to reach the site of action (“systemic delivery”). The present invention finds application in both local delivery (i.e., treatment of skin itself) and systemic delivery of therapeutic agents. Delivery of therapeutic agents with the present invention can result in immediate as well as sustained or prolonged therapeutic activity depending upon the characteristics of the drug and its formulation.

In the case of local delivery, drug is deposited into the dermal layers and exerts its activity at or near the site of deposition. Drugs suitable for local administration to the skin include local anesthetics and drugs which treat dermatological conditions. Examples include, but are not limited to, drugs (and their salts and derivatives) classified as:

Analgesics, for example, aspirin;

Antipuretics, for example Diphenhydramine and Hydroxyzine;

Antibiotics, for example, Clindamycin, Mupirocin, Erythomycin, Ceflasporin and Benzoyl Peroxide;

Antifungals, for example, Ciclopirox, Clortrimazole, Miconazole, Butenafine, Naftin, Ketoconazole, Oxiconazole nitrate, Metronidazole, Itraconazole, Amphoterican B, Nystatin, Flucytosin, Natamycin, Econazole, Griseofulvin, Vorixonazole, and Terconazole;

Anti-inflammatories, for example, diclofenac;

Antivirals, for example, Penciclovir, Acyclovir and Pimecrolimus;

Antineoplastics, for example, Fluorouracil;

Antipsoriatic, for example, Calcipotriene, Cyclosporine, Acitretin, and Tazarotene;

Anti-seborrheic agents;

Agents to treat bums, for example, Silver sulfadiazine and Mafenide acetate;

Cosmetic Agents, for example, Botulinum Toxin Type A and Collagen;

Depigmenting agents, for example, Hydroquinone and Monobenzone;

Hair Growth Retardants, for example, Eflornithine;

Hair Growth stimulants, for example, Minoxidil and Finasteride;

Retonoids, for example, Tazarotene and Adapaline;

Local anesthetics, for example, lidocaine, ropivacaine, procaine, tetracaine, prilocaine, amethocaine, benzocaine, butamben, dibucaine, dimethisoquin, diperodon, ketocaine, pramoxine, propanocaine, propipocaine, proxycaine, and bupivacaine;

Pigmentation agents; and

Steroids, for example, Clobetasol propionate, Diflorasone diacetate, Halbetosal propionate, Amcinonide, Dexoximethasone, Fluocinonide, Halocinonide, Mometasone furoate, Triamcinolone acetonide, Amicinonide, Fluticasone propionate, Triamcinolone acetate, Fluocinolone acetonide, Flurandrenolide, Fluticasone propionate, Hydrocortisone valerate or acetate, Mometasone furoate, Clocortolone private, Flurandrenolide, Fluticasone propionate, Hydrocortisone butyrate or probutate, Predincarbate, Aclometasone dipropionate, Desonide, Dexamethasone, Hydrocortisone, and Methylprednisolone.

The present invention may find application in the delivery of local anesthetics, such as lidocaine, ropivacaine, procaine, tetracaine, prilocaine, and bupivacaine. Current methods to achieve local anesthesia over an area of skin (such as creams, ointments and iontophoresis) are associated with a significant lag time in therapeutic onset. The present invention may provide a rapid onset of anesthesia over a defined dermal region in a less painful and threatening manner than traditional injections with a standard needle and syringe can provide.

Another local application of the present invention is the delivery of steroids through the scar tissue that may form during surgery, cosmetic surgery in particular. The present therapy is to deliver steroids at the location where the scar tissue begins to form.

In the case of systemic delivery, drug is deposited at an appropriate depth within or below the skin, thus bypassing the stratum corneum (known to be the primary barrier to transdermal drug delivery) and is subsequently absorbed into the circulation. Drugs suitable for systemic administration via the present invention include those therapeutic agents that are currently administered via injection and many agents that are traditionally administered orally or via transdermal patch. Examples of suitable agents include, but are not limited to, the following agents (and their salts and derivatives) classified as:

Alzheimer's Disease treatment, for example, donepezil;

Antibiotic agents, for example, cefoperazone, cefotaxime, ceftizoxime, cefepime;

Anti-emetic agents, for example, granisetron, prochlorperazine, trimethobenzamide;

Anti-epileptic agents, for example, valproic acid, ketorolac tromethamine, phenytoin, lamotrigine;

Anti-pyretics and analgesics, for example, aspirin;

Cardiac treatment, for example, eptifabatide, enoxaparin

Contraceptive agents, for example, progesterone, estradiol

Deep Vein Thrombosis Prophylaxis, for example, fondaparinux sodium, heparin sodium, dalteparin sodium

Diagnostic Agents, for example, tuberculin purified protein derivative, gonadorelin hydrochloride

Hemophilia treatment, for example, coagulation factor VIIa

Hepatitis C treatment, for example, interferon alpha 2b, peg-interferon alpha 2b, interferon alpha 2a

HIV/AIDS treatment, for example, lamivudine, somatropin;

Hormones, for example, testosterone, estrogen, progesterone;

Immunosupressants, for example, imiglucerase, cyclosporine;

Infertility treatment, for example, follitropin alpha, ganirelix acetate, cetrorelix acetate, choriogonadotropin alpha, follitropin beta, urofollitropin, menotropins;

Insomnia treatment, for example, zolpidem;

Migraine treatment, for example, sumatriptan;

Multiple sclerosis treatment, for example, glatiramer acetate, interferon beta 1a;

Osteoporosis treatment/prevention, for example, alendronate sodium, interferon gamma 1b;

Pain management, for example, fentanyl, morphine, oxycodone, meperidine, hydromorphone, nalbuphine hydrochloride;

Parkinson's Disease treatment, for example, rotigotine, selegiline, levodopa/carbidopa;

Psychiatric drugs, for example, lithium, danzapine, bupropion, risperidone, milnacipran;

Rheumatoid arthritis treatment, for example, diclofenac diflusinal, methotrexate, etanercept, anakinra;

Vaccines, for example, Comvax® (haemophilis B conjugate and hepatitis B vaccine), hepatitis B vaccine, Deptacel® (diphtheria and tetanus toxoids and acellular pertussis vaccine adsorbed), rabies vaccine, hepatitis A vaccine, pneumococcal vaccine polyvalent, poliovirus vaccine inactivated, Japanese encephalitis vaccine inactivated, varicella virus vaccine live, measules, mumps, and rubella virus vaccine, measles virus vaccine live, yellow fever vaccine;

Vitamins, for example, vitamin K, vitamin B12;

Protein and Peptide therapeutics (and their derivatives) for various indications, for example, insulin, glucagons, heparin, leuprolide, erythropoietin, calcitonin, desmopressin acetate, octreotide acetate, interferon, sargramostin, leuprolide acetate, somatropin, interleukin, oprelvekin, pegfilgrastim, secretin;

Monoclonal antibodies, for example, palivizumab (Synagis®), alemtuzumab (Campath®), infliximab, muromonab-CD3 (Orthoclone OKT3®);

Other miscellaneous drugs include, for example, naloxone, epinephrine, Traumeel® (botanical mix), metaraminol bitartrate, bethanechol chloride, micrurus fulvius, crotalidae polyvalent, black widow spider anti-venom, hydroxyzine.

The current invention combines the advantages of three methods currently used to administer therapeutic compounds: topical creams and ointments; transdermal patches; and injection with a traditional needle and syringe. Like traditional injections with a needle and syringe, the present invention bypasses the primary barrier to absorption through the skin, the thin layer of dead cells known as the stratum corneum. Consequently, a given therapeutic compound will reach its site of action more quickly and efficiently than with when applied as a topical cream, ointment, or patch. This is advantageous when a rapid therapeutic onset from time of drug delivery is required or for compounds that do not penetrate the outermost layer of skin in therapeutically effective levels. Furthermore, like ointments and creams, it has the advantage of being able to cover an arbitrary area of skin. Finally, in contrast to delivery via transdermal patch, which is severely limited by the dose required and the physio-chemical properties of the compound (i.e., molecular weight, pKa, and octanol water partition coefficient), delivery via the present invention provides a transdermal route for those drugs that are not capable of penetrating the skin passively at a desired rate.

The present invention has the potential to overcome the inherent delivery limitations of certain therapeutic agents. For example, although many drugs are administered orally due to the convenience of this route, the oral route is not desirable or possible for some therapeutic agents. In some instances therapeutic compounds are destroyed, inactivated or metabolized when delivered orally due to the low pH environment of the stomach or the enzymatic activity within the GI tract. This results in poor efficacy or unwanted side effects. In these cases an alternative delivery method is often sought and injections (intramuscular, intravenous or subcutaneous) are often used. Additionally, oral delivery is not optimal for certain patient populations and conditions; in fact, it is contra-indicated for those patients who are vomiting, unconscious, or suffer from seizures. Delivery via the present invention is suitable for avoiding the pain and/or inconvenience of traditional injections with a standard needle and syringe. For example, many proteins, hormones and nucleic acid based therapeutics, which are commonly degraded or poorly absorbed when delivered orally, are ideal candidates for administration with the present invention. Likewise, small molecule therapeutics that show poor efficacy when delivered orally due to extensive first pass metabolism, poor absorption, or low aqueous solubility may show enhanced efficacy when injected via the present invention. Additionally, a lower dose may be administered since bypassing the GI tract will increase the bioavailability of such compounds.

The oral administration of medicaments is problematic for other reasons, such as difficulty in oral administration. One application of the disclosed systems and methods would be the delivery of medicaments to newborn or young children, Alzheimer's patients who have forgotten how to swallow, individuals with mouth, throat, or stomach ailments, or persons for whom oral administration of any medicament is difficult or problematic.

The present invention is suited for applications where delivery into the skin, rather than below the skin, is required. For example, the efficacy of many vaccine antigens may be potentiated by injecting them into the viable epidermis, since this region of the skin is known to be rich in antigen presenting cells (Langerhans cells). In contrast to injection via traditional needle and syringe which requires a skilled and trained professional, the present invention allows for reproducible intradermal injection. Thus, the present invention provides a more convenient method to achieve injection within the skin layers and is thus particularly well suited for delivery of vaccine antigens.

The duration of treatment achieved by a therapeutic agent following administration via the present invention may be prolonged by incorporating the drug of interest into a carrier that produces a slow release of drug over time within the dermal layers. Liquid and solid compositions capable of controlled release drug delivery as known in the pharmaceutical arts may be injected with the present invention. In this way, release durations of hours to months may be achieved by an appropriate drug-carrier composition. For example, biodegradable carriers in the form of microspheres, nanospheres, or injectable gels allow for controlled release “depots” of drug to be deposited below or within the skin. Materials suitable for this purpose are known in the pharmaceutical arts, see for example Cleland, J. L., et al., Current Opinions in Biotechnology, 2001, Vol. 12, pp. 212-219; Sinha, V. R., et al., Journal of Controlled Release, 2003, Vol. 90, pp. 261-280; and Hatefi, A., et al., Journal of Controlled Release, 2002, Vol. 80, pp. 9-28, incorporated by reference herein. Examples of materials that may be suitable carriers to produce a controlled release of drug include, but are not limited to, polyanhydrides, polyesters (e.g., polylactides and copolymers), polyester derivatives, poly(orthoesters), photopolymerizable hydrogels, sucrose acetate isobutyrate, lipid foams, collagen, alginates, and hyaluronic acid derivatives.

In addition to the microspheres, nanospheres and injectable gels listed above, controlled release may also be achieved following injection of appropriately formulated aqueous solutions or suspensions; oil solutions or suspensions; or oil and water based emulsions. (See “Remington, The Science and Practice of Pharmacy”, 20^{th} Edition, pages 914-916, incorporated by reference herein). A degree of controlled release can be imparted on aqueous solutions by the addition of thickening agents to increase the viscosity of aqueous solutions (examples include, but are not limited to, methylcellulose, sodium carboxymethylcellulose and polyvinylpyrolidone). Furthermore, drug may be modified to achieve a relatively slow absorption rate, and thus a sustained therapeutic effect. This includes the use of less water-soluble salts; complexes with biological or synthetic polymers; and less soluble polymorphic forms. Modified drugs may be formulated in aqueous suspensions or, alternatively, as a suspension in an oil such as sesame, olive, archnis, maize, almond, cottonseed or castor oil.

The present invention is suitable for applications where painless delivery of the medicament is preferred. The range of such applications includes: the administration of medicaments to newborn or young children, the elderly, or persons with low pain thresholds; administration of medicaments to particularly sensitive parts of the body; and administration of medicaments in a setting where pain or fear of needles would be a significant deterrent to treatment. In certain patient populations adverse to injection, injections with the present invention may be incorporated into routine physical manipulations, such as into a massage treatment.

The present invention may provide targeted delivery of the medicament. As mentioned above in the description, the device may comprise a sensor system. The sensor system could distinguish between areas of the skin with different characteristics and deliver the medicament at designated points. For example, a care provider may mark points or regions of the skin for treatment using ink or dye. The sensor system may also detect naturally occurring characteristics of a particular condition and deliver medicament. Examples of such conditions include shingles, poison ivy, burns, or scar tissue. The sensor system may be used to detect the edges of a tattoo and deliver a medicament to dilute or blur the tattoo.

In other embodiments of the present invention, the force or the depth of penetration is adjustable and controlled by mechanical or non-mechanical means. The force may be controlled as a function of the speed at which the perforator penetrates through the skin. The depth of penetration may be controlled by the length of the perforator, or by other mechanical or non-mechanical means. One example of a mechanical means may be a protrusion such as stop tab (not shown) that prevents the reservoir and perforator from traveling deeper into the skin. Controlling the force or the depth of penetration allows adjustments to the depth of penetration and the volume of penetration for each patient. For example, some patients have less fat than others, and therefore the force of opposition to penetration of the perforator varies. In particular, many elderly patients have a very reduced amount of fat, therefore, the force required for a perforator to penetrate the skin to a desired depth is reduced. Furthermore, the depth of penetration may be selectively controlled to deliver medicament intramuscularly, subcutaneously, or otherwise. In one aspect, the system takes advantage of the fact that the volume of the therapeutic in the perforator is injected as a function of the kinetic energy applied by the flexure to make this adjustment.

It is anticipated that the present invention is effective in administering liquid based formulations of varying viscosities, including oils, emulsions and suspensions. In some embodiments it may be advantageous to incorporate ultrasonic means to maintain a uniform suspension or emulsion during a treatment. It is further anticipated that the present invention is effective in administering liquids of varying temperatures; this may prove advantageous as changing the temperature provides a method to effect the flow properties of liquids.

### EQUIVALENTS

Those skilled in the art will recognize, or be able to ascertain, using no more than routine experimentation, numerous equivalents to the specific mechanisms, devices, configurations, materials, and medicaments described herein. Such equivalents are considered to be within the scope of this invention.

What is claimed is:

1. A medicament delivery system comprising:
a reservoir for storing a medicament,
a channel for carrying a volume of the medicament from the reservoir into a passage within a perforator, and
an actuator coupled to the perforator and capable of applying a decelerating force thereto for causing the inertia of the volume to eject the medicament from the hollow perforator.

2. The device of claim 1 further comprising a flexure mounted on the actuator and connected to the reservoir.

3. The device of claim 1 wherein the actuator comprises a magnetic coil.

4. The device of claim 1 further comprising a housing having an aperture disposed therein.

5. The device of claim 4 wherein the housing further comprises a bulge area surrounding the aperture.

6. The device of claim 5 wherein said bulge area is made of a material selected from the group of glass, polymer, metal, ceramic, and composite.

7. The device of claim 5 wherein the bulge area is configured to stretch the skin at its point of contact.

8. The device of claim 7 wherein the bulge area comprises flanges or a circular ridge.

9. The device of claim 1 wherein said reservoir further comprises a gas source.

10. The device of claim 9 wherein said gas source provides pressure to supplement the inertia utilized to deliver the medicament.

11. The device of claim 1 wherein said reservoir is made from a material is selected from the group of glass, polymer, metal, ceramic, and composite.

12. The device of claim 1 wherein said reservoir has a volume of about 10 microliters to about 50 milliliters.

13. The device of claim 1 wherein said reservoir further comprises a relief valve.

14. The device of claim 1 wherein said perforator is a microtube.

15. The device of claim 1 wherein said perforator has a volume of about 0.5 nanoliter to about 10 microliters.

16. The device of claim 1 wherein said perforator has a length of about 200 micrometers to about 5 millimeters.

17. The device of claim 1 wherein said perforator has an orifice of about 20 micrometers to about 800 micrometers in diameter.

18. The device of claim 1 wherein said flexure is a membrane.

19. The device of claim 18 wherein the membrane is made of a material selected from diamond coated titanium, rubber, latex, metal, polymer, and ceramic.

20. The device of claim 1 wherein the device comprises a plurality of perforators.

21. The device of claim 1 wherein the device comprises a plurality of reservoirs in fluid connection with at least one perforator.

22. The device of claim 1 further comprising a sensor for determining the position of the flexure.

23. The device of claim 1 further comprising a sensor for determining the position of the perforator.

24. The device of claim 1 further comprising a sensor for determining the position of the device along the surface of the skin.

25. The device of claim 24 wherein the sensor is selected from the group of an optical sensor, an impedance sensor, a temperature sensor, and a pH sensor.

26. The device of claim 1 wherein said actuator activates the flexure by a piezoelectric, magnetostriction, magnetic, electromagnetic, mechanical, hydraulic, or pneumatic means.

27. The device of claim 1 wherein the reservoir is connected to the perforator by a length of flexible tubing.

28. The device of claim 1 wherein said medicament is selected for local delivery.

29. The device of claim 28 wherein said medicament is selected to treat a skin disorder.

30. The device of claim 28 wherein said medicament is a local anesthetic.

31. The device of claim 28 wherein said medicament is selected from the group of analgesics, antipuretics, antibiotics, antifungals, anti-inflammatories, antivirals, antineoplastics, antipsoriatic, anti-seborrheic agents, agents to treat burns, cosmetic agents, depigmenting agents, hair growth retardants, hair growth stimulants, retinoids, local anesthetics, pigmentation agents, and steroids.

32. The device of claim 1 wherein said medicament is selected for systemic delivery.

33. The device of claim 32 wherein said medicament is selected from the group of Alzheimer's Disease agents, antibiotic agents, Anti-emetic agents, Anti-epileptic agents, Anti-pyretic agents, analgesics, Cardiac treatment agents, Contraceptive agents, Deep Vein Thrombosis Prophylaxis agents, Diagnostic Agents, Hemophilia treatment agents, Hepatitis C treatment agents, HIV/AIDS treatment agents, Hormones, Immunosupressants, Infertility treatment agents, Insomnia treatment agents, Migraine treatment agents, Multiple sclerosis treatment agents, Osteoporosis treatment/prevention agents, Pain management agents, Parkinson's Disease treatment agents, Psychiatric drugs, Rheumatoid arthritis treatment agents, Vaccines, Vitamins, Protein and Peptides, nucleic acid molecules, and Monoclonal antibodies.

34. The device of claim 1, wherein the medicament provides a slow release of drug over time.

35. The device of claim 34, wherein the medicament comprises a carrier selected from polyanhydrides, polyesters, polyester derivatives, poly(orthoesters), photopolymerizable hydrogels, sucrose acetate isobutyrate, lipid foams, collagen, alginates, hyaluronic acid derivatives, methylcellulose, sodium carboxymethylcellulose and polyvinylpyrolidone.

36. The device of claim 34, wherein the medicament is formulated in aqueous suspensions or suspensions in oil.

37. The device of claim 28-33, wherein the medicament provides a slow release of drug over time.

38. The device of claim 28-33, wherein the medicament comprises a carrier selected from polyanhydrides, polyesters, polyester derivatives, poly(orthoesters), photopolymerizable hydrogels, sucrose acetate isobutyrate, lipid foams, collagen, alginates, hyaluronic acid derivatives, methylcellulose, sodium carboxymethylcellulose and polyvinylpyrolidone.

39. The device of claim 28-33, wherein the medicament is formulated in aqueous suspensions or suspensions in oil.

40. A method of delivering medicament comprising:
providing a medicament in a reservoir in fluid connection with a hollow perforator, and
accelerating said perforator followed by abrupt deceleration such that the volume of medicament is ejected into the skin.

41. The method of claim 40 wherein the method is employed to deliver medicament during massage.

42. A method of manufacturing a drug delivery device comprising the steps of:
providing a reservoir for storing a medicament,
providing a channel for carrying a volume of medicament from the reservoir into a passage within a perforator, and
providing an actuator coupled to the perforator and capable of applying a decelerating force thereto for causing inertia of the volume to eject the fluid from the hollow perforator.
